(19) United States
(12) Patent Application Publication
(10) Pub. No.: US 2005/0268405 A1
(43) Pub. Date: Dec. 8, 2005

(54) Composition for dyeing keratin fibers, comprising at least one pigment and polymers capable of reacting with each other to form covalent bonds

(76) Inventors: Gaelle Brun, Paris (FR); Gabin Vic, Venette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/139,675

(22) Filed: May 31, 2005

Related U.S. Application Data
(60) Provisional application No. 60/580,100, filed on Jun. 17, 2004.

(30) Foreign Application Priority Data
May 28, 2004 (FR) 04 05834

(51) Int. Cl. A61K 7/13

(52) U.S. Cl. 8/405

(57) ABSTRACT

The present disclosure relates to compositions for dyeing keratin fibers comprising at least one pigment, at least one polymer PA bearing at least one functional group A, and at least one polymer PB bearing at least one functional group B, wherein the functional groups A and B can form covalent bonds together; to processes for dyeing keratin fibers with the compositions as disclosed herein, and also to the use of this pigment and of these polymers for dyeing keratin fibers in a color-fast manner.

This application claims benefit of U.S. Provisional Application No. 60/580,100, filed Jun. 17, 2004, the contents of which are incorporated herein by reference. This application also claims benefit of priority under 35 U.S.C. § 119 to French Patent Application No. 04 05834, filed May 28, 2004, the contents of which are also incorporated herein by reference.

The present disclosure relates to a composition for dyeing keratin fibers, for instance human keratin fibers such as the hair, comprising and at least one pigment, at least one polymer PA and at least one polymer PB, wherein the at least one polymer PA and at least one polymer PB can react with each other to form covalent bonds.

In the field of dyeing keratin fibers, there are two modes of dyeing, which each have their advantages and their drawbacks.

Direct dyeing or semi-permanent dyeing comprises dyeing keratin fibers with dye compositions comprising direct dyes. These dyes are colored and they are coloring molecules that have affinity for keratin fibers. They are applied to the keratin fibers for a time necessary to obtain the desired coloration, and are then rinsed out.

The standard dyes that can be used include, for example, dyes of the nitrobenzene, anthraquinone, nitropyridine, azo, xanthene, acridine, azine or triarylmethane type or natural dyes.

It has been proposed in the past to use pigments, for instance in French Patent Application No. FR 2 741 530, which envisages, for dyeing keratin fibers, the use of a composition comprising at least one dispersion of particles of film-forming polymer comprising at least one acid functional group and at least one pigment dispersed in the continuous phase of the dispersion.

The colorations obtained via direct dyeing can have the drawback of having low color-fastness, and may fade, for example, from shampooing.

Oxidation dyeing or permanent dyeing comprises dyeing keratin fibers with dye compositions comprising oxidation dye precursors, which are also generally known as oxidation bases, such as ortho- or para-phenylenediamines, ortho- or para-aminophenols and heterocyclic compounds such as diaminopyrazole derivatives. These oxidation bases are colorless or weakly colored compounds, which, when combined with oxidizing products, can give rise to colored compounds via a process of oxidative condensation.

It is also known that the shades obtained with these oxidation bases can be varied by combining them with couplers or coloration modifiers, the latter can be chosen from, for example, aromatic meta-diamines, meta-aminophenols, meta-diphenols and certain heterocyclic compounds such as indole compounds.

The shades obtained via oxidation dyeing generally show relatively good color-fastness.

The difficulty with these two modes of dyeing, i.e., direct dyeing or semi-permanent dyeing and oxidation dyeing or permanent dyeing, is that if it is desired to dye a dark support, it is necessary to bleach or lighten the support in order for the supplied color to be visible.

The bleaching or lightening of keratin fibers may be performed using an oxidizing agent. Among the conventionally used oxidizing agents that may be mentioned include hydrogen peroxide or compounds capable of producing hydrogen peroxide by hydrolysis, for instance urea peroxide or persalts such as perborates, persulfates and percarbonates.

The use of oxidizing agents, whether to lighten and/or to oxidize oxidation dyes, can have the drawback of resulting in appreciable degradation of the keratin fibers and of impairing their cosmetic properties. The hair has a tendency to become coarse, more difficult to disentangle and more brittle.

Accordingly, the present disclosure relates to novel compositions for dyeing keratin fibers, for instance human keratin fibers such as the hair, which allow colorations to be obtained that are visible on a dark support without it being necessary to lighten or bleach the fibers, and that show good color-fastness.

Thus one aspect of the present disclosure is a composition for dyeing keratin fibers, comprising, in a cosmetically acceptable medium, at least one pigment, at least one polymer PA bearing at least one chemical functional group A, and at least one polymer PB bearing at least one chemical functional group B, wherein the chemical functional groups A and B can form covalent bonds together.

The composition in accordance with the present disclosure can lead to a visible coloration on a dark support without it being necessary to lighten or bleach the keratin fibers, and consequently without any physical degradation of the keratin fibers. This coloration, furthermore, can be resistant to the various attacking factors to which the hair may be subjected, such as shampoos, rubbing, light, bad weather, sweat and permanent reshaping operations. These properties can be particularly noteworthy regarding the resistance of the coloration with respect to shampoos and rubbing. The coloration can be obtained in varied shades and can be chromatic, strong, aesthetic and sparingly selective.

The present disclosure also relates to a process for dyeing keratin fibers comprising applying to the keratin fibers a composition comprising at least one pigment, at least one polymer PA bearing at least one chemical functional group A, and at least one polymer PB bearing at least one chemical functional group B, wherein the functional groups A and B can react with each other to form covalent bonds.

The present disclosure still further relates to the use, for dyeing keratin fibers, of at least one pigment, at least one polymer PA bearing at least one chemical functional group A, and at least one polymer PB bearing at least one chemical functional group B, wherein the functional groups A and B can react with each other to form covalent bonds.

In accordance with the present disclosure, in order to determine whether the chemical functional groups A and B of the at least one polymer PA and at least one polymer PB react with each other to form covalent bonds, for example by substitution reaction, addition reaction to carbon-carbon or carbon-hetero atom double or triple bonds, or ring-opening reaction, the test described below is performed, wherein steps (1) to (4) are performed at room temperature:
(1) 0.25 gram of a solution of the polymer PA in at least one solvent chosen from water, ethanol, esters and ketones, such as water, wherein the polymer PA is present in an amount ranging from 10% to 50% by weight, relative to the total weight of the solution, is applied to a glass slide;
(2) the solvent of the polymer PA is allowed to evaporate off, until a dry deposit is obtained;
(3) 0.25 gram of a solution of the polymer PB in at least one solvent chosen from water, ethanol, esters and ketones, such as water, wherein the polymer PB is present in an amount ranging from 10% to 50% by weight, relative to the total weight of the solution, is applied to the slide already coated with the polymer PA;
(4) the solvent of the polymer PB is allowed to evaporate off, until a dry deposit is obtained;
(5) the glass slide coated with the polymers PA and PB is placed in a chamber at a temperature of 130° C., for a period of 120 minutes;
(6) when the solvents used in steps (1) and (3) are different, steps (1) to (5) are repeated a second time;
(7) the solid obtained is totally immersed in 10 grams of the solvent used in step (1);
(8) when the solvents used in steps (1) and (3) are different, the solid obtained from step (6) is immersed into 10 grams of the solvent used in step (3);
(9) the polymers PA and PB react with each other to form covalent bonds if at least 50% by weight of the solid does not dissolve after three days, at room temperature and without stirring, in the solvents of steps (7) and (8).

In steps (1) and (3), when the solutions are aqueous, they can be, for example, adjusted to the working pH of the compositions of the present disclosure.

The formation of covalent bonds between the polymers PA and PB may also be checked by means of standard characterization techniques, such as infrared spectroscopy or X-ray photoelectron spectroscopy (XPS).

In the context of the present disclosure, the chemical functional groups A can react with the chemical functional groups B either spontaneously or via activation by temperature, pH, a co-reagent or a chemical or biochemical catalyst, for instance an enzyme.

According to one aspect of the present disclosure, the chemical functional groups A can be chosen from the functional groups:
epoxide,
aziridine,
vinyl and activated vinyl, for instance acrylonitrile, acrylic and methacrylic esters, crotonic acid and esters, cinnamic acid and esters, styrene and derivatives, butadiene, vinyl ethers, vinyl ketone, maleic esters, vinyl sulfones and maleimides,
carboxylic acid anhydride, acid chloride and esters,
aldehydes,
acetals, hemiacetals,
aminals, hemiaminals,
ketones, α-hydroxy ketones, α-halo ketones,
lactones, thiolactones,
isocyanate,
thiocyanate,
imines,
imides, such as succinimide and glutimide,
N-hydroxysuccinimide esters,
imidates,
thiosulfate,
oxazine and oxazoline,
oxazinium and oxazolinium,
C₁ to C₃₀ alkyl halides or C₆ to C₃₀ aryl or aralkyl halides of formula RX with X=I, Br or
halide of a carbon-based unsaturated ring or heterocycle, for instance chlorotriazine, chloropyrimidine, chloroquinoxaline or chlorobenzotriazole,
sulfonyl halides of formula RSO₂C₁ or F, R being chosen from C₁ to C₃₀ alkyl groups.

In one embodiment of the present disclosure, the chemical functional groups A are chosen from anhydride, epoxide, chlorotriazine, aldehyde and thiosulfate functional groups.

According to one aspect of the present disclosure, the chemical functional groups B are chosen from hydroxyl, primary and secondary amine, thiol and carboxylic acid functional groups.

The polymers according to the present disclosure may comprise chemical functional groups other than the functional groups of A and B.

As used herein, the term “polymer” is understood to mean a compound comprising at least two repeating units linked together via covalent bonds.

The at least one polymer PA and at least one polymer PB may be of natural origin, chemically modified or otherwise, for instance polysaccharides such as cellulose, dextran, chitosan and guar, and hydroxyalkyl, carboxymethyl, amino or thiol derivatives thereof, or derivatives thereof comprising an aldehyde or epoxy functional group.

They may be synthetic polymers such as polyacrylates, polymethacrylates, polyvinyls, polyesters, polyethers, polyamides, polyurethanes, polydimethylsiloxanes or polypeptides. These polymers may be synthesized according to methods known in the literature.

These polymers may have any type of topology, including linear, branched, star or hyperbranched chain, such as dendrimers, and block, random or alternating chains.

The chemical functional groups A and B may be present on the polymer chain, at the end of a chain, grafted along the main chain or side chains, or on the branches of star or hyperbranched polymers.

In one embodiment of the present disclosure, the at least one polymer PA comprises at least two identical chemical functional groups A and the at least one polymer PB comprises at least two identical chemical functional groups B, so as to bond with at least two other polymers.

By way of non-limiting example, the at least one polymer PA can be chosen from:
polymers comprising an anhydride unit, such as maleic anhydride,
polymers comprising an epoxide and/or aldehyde group,
natural or modified polysaccharides comprising aldehyde functional groups,
natural or modified polysaccharides comprising epoxy functional groups, and
natural or synthetic polymers comprising carboxylic acid functional groups.

The at least one polymer PB can be chosen from, by way of non-limiting example, synthetic or natural polymers comprising OH, NH₂, SH or COOH functional groups.

As examples of synthetic or natural polymers comprising OH, NH₂, SH or COOH functional groups, non-limiting mention may be made of:
dendrimers comprising OH, NH₂, SH or COOH end groups of at least one generation,
synthetic polymers comprising a hydroxyl functional group, such as polyvinyl alcohols,
polyethyleneimines,
polyethyleneimine thiols obtained by reacting polyethyleneimines with γ-butyrolactone, such as those described in French Patent No. FR 2 772 770,
polyamino acids comprising free OH, NH₂, SH or COOH groups, for example polylysine,
natural or modified polysaccharides comprising OH, NH₂, SH or COOH functional groups, for instance chitosan and derivatives thereof, such as carboxymethylchitosans or aminodextrans.

According to the present disclosure, non-limiting examples of the combinations of the least one polymer PA and at least one polymer PB include:
at least one polymer PB chosen from dendrimers comprising OH, NH₂, SH or COOH end groups of at least one generation in combination with at least one polymer PA chosen from polymers comprising an anhydride unit, such as maleic anhydride, or polymers comprising an epoxide and/or aldehyde group,
at least one polymer PB chosen from synthetic polymers comprising a hydroxyl functional group, such as polyvinyl alcohols, in combination with at least one polymer PA chosen from polymers comprising an anhydride unit, such as maleic anhydride, or polymers comprising an epoxide group,
at least one polymer PB chosen from polyethyleneimines in combination with at least one polymer PA chosen from polymers comprising an anhydride unit, such as maleic anhydride, or polymers comprising an epoxide and/or aldehyde group,
at least one polymer PB chosen from polyethyleneimine thiols, obtained by reacting polyethyleneimines with γ-butyrolactone, such as those described in French Patent No. FR 2 772 770, in combination with at least one polymer PA chosen from polymers comprising an anhydride unit, such as maleic anhydride, or polymers comprising an epoxide and/or aldehyde group,
at least one polymer PB chosen from polyamino acids comprising free OH, NH₂, SH or COOH functional groups, for example polylysine in combination with at least one polymer PA chosen from polymers comprising an anhydride unit, such as maleic anhydride, or polymers comprising an epoxide and/or aldehyde group,
at least one polymer PB chosen from natural or modified polysaccharides comprising OH, NH₂, SH or COOH functional groups in combination with at least one polymer PA chosen from polymers comprising an anhydride unit, such as maleic anhydride, or polymers comprising an epoxide and/or aldehyde group,
at least one polymer PA chosen from natural or modified polysaccharides comprising aldehyde functional groups in combination with at least one polymer PB chosen from synthetic or natural polymers comprising OH, NH₂, SH or COOH functional groups, for instance polyethyleneimine, polylysine, chitosan and its derivatives, such as carboxymethylchitosans or aminodextrans,
at least one polymer PA chosen from natural or modified polysaccharides comprising epoxy functional groups in combination with at least one polymer PB chosen from synthetic or natural polymers comprising OH, NH₂, SH or COOH functional groups, for instance polyethyleneimine, polylysine, chitosan and its derivatives, such as carboxymethylchitosans, or aminodextrans,
at least one polymer PA chosen from natural or synthetic polymers comprising carboxylic acid functional groups in combination with at least one polymer PB chosen from synthetic or natural polymers comprising OH, NH₂ or SH functional groups, in the presence of a carbodiimide, or of an acid, a base or an enzyme, for instance esterases, lipases or proteases.

Examples of the polymers PA that may be mentioned, in a non-limiting manner, include:
methyl vinyl ether/maleic anhydride copolymer, such as sold, for example, by ISP under the name Gantrez,
polyglycidyl methacrylate, for instance, sold by Polysciences,
glycidyl polydimethylsiloxane, such as sold by Shin-Etsu (reference X-2Z-173 FX or DX),
epoxy polyamidoamine, for example sold by Hercules under the name Delsette 101, or Kymene 450 from Hercules,
epoxydextran,
polyaldehyde polysaccharides obtained by oxidation of polysaccharides with NaIO₄ (known methods, Bioconjugate Techniques; Hermanson G T, Academic Press, 1996).

Examples of the polymers PB that may be mentioned, in a non-limiting manner, include:
PAMAM dendrimer, for instance sold by Dendritech, DMS, Sigma-Aldrich (Starburst, PAMAM Dendrimer, G(2,O) from Dendritech),
dendrimer comprising hydroxyl functional groups, for instance sold by Perstorp, DSM (example: HBP TMP core 2 Generation Perstorp),
PEI (polyethyleneimine), for instance sold by BASF under the name Lupasol,
PEI-thiol,
polylysine, for example, sold by Chisso,
HP cellulose, such as Klucel EF from Aqualon,
aminodextran, for example sold by Carbomer,
aminocellulose, for example those described in International Patent No. WO 01/25283 from BASF,
PVA (polyvinyl acetal), for example Airvol 540 from Air Products Chemical,
Amino PVA, for example sold by Carbomer,
chitosan,
CM or HP dextran, for example sold by Fluka,
CM or HP chitosan, for example sold by Fluka.

All of the combinations of at least one polymer PA comprising chemical functional groups A and of at least one polymer PB comprising chemical functional groups B described herein are mentioned as non-limiting examples.

As used herein, the term “pigment” is understood to mean any organic and/or mineral species whose solubility in water is less than 0.01% at 20° C., having an absorption ranging from 350 nm to 700 nm, such as an absorption with a maximum.

The at least one pigment in accordance with the present disclosure can be chosen from all organic and/or mineral pigments, such as those described in Kirk-Othmer's Encyclopaedia of Chemical Technology and in Ullmann's Encyclopaedia of Industrial Chemistry.

The at least one pigment in accordance with the present disclosure may be in the form of powder or of pigmentary paste. They may be coated or uncoated. The at least one pigment in accordance with the present disclosure can be chosen, for example, from white and colored pigments, lakes, and pigments with special effects such as nacres and flakes.

Non-limiting examples of white or colored mineral pigments that may be mentioned include titanium dioxide, which may or may not be surface-treated, zirconium oxide or cerium oxide, iron oxide or chromium oxide, manganese violet, ultramarine blue, chromium hydrate and ferric blue. Further non-limiting examples of white or colored organic pigments that may be mentioned include nitroso, nitro, azo, xanthene, quinoline, anthraquinone and phthalocyanin compounds, compounds of metallic complex type, and isoindolinone, isoindoline, quinacridone, perinone, perylene, diketopyrrolopyrrole, thioindigo, dioxazine, triphenylmethane and quinophthalone compounds.

For instance, the white or colored organic pigments can be chosen from carmine, carbon black, aniline black, azo yellow, quinacridone, phthalocyanin blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in French Patent No. FR 2 679 771.

Pigmentary pastes of at least one organic pigment can also be used, such as the products sold by the company Hoechst under the name:
Jaune Cosmenyl IOG: Pigment Yellow 3 (CI 11710);
Jaune Cosmenyl G: Pigment Yellow 1 (CI 11680);
Orange Cosmenyl GR: Pigment Orange 43 (CI 71105);
Rouge Cosmenyl R: Pigment Red 4 (CI 12085);
Carmin Cosmenyl FB: Pigment Red 5 (CI 12490);
Violet Cosmenyl RL: Pigment Violet 23 (CI 51319);
Bleu Cosmenyl A2R: Pigment Blue 15.1 (CI 74160);
Vert Cosmenyl GG: Pigment Green 7 (CI 74260); and
Noir Cosmenyl R: Pigment Black 7 (CI 77266).

The at least one pigment in accordance with the present disclosure may also be in the form of composite pigments as described in European Patent No. EP 1 184 426. The composite pigments may comprise, for example:
a mineral core,
at least one binder for fixing the organic pigments to the core, and
at least one organic pigment at least partially covering the core.

As used herein, the term “lakes” is understood to mean dyes adsorbed onto insoluble particles, the assembly thus obtained remaining insoluble during use. The mineral substrates onto which the dyes are adsorbed can be, for example, alumina, silica, calcium sodium borosilicate or calcium aluminium borosilicate and aluminium. Among the organic dyes that may be used, non-limiting mention may be made of cochineal carmine.

Examples of lakes that may be mentioned, in a non-limiting manner, include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), and D & C Blue 1 (CI 42 090).

As used herein, the term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). Such pigments are, consequently, in contrast with white or colored pigments, which afford a standard opaque, semi-transparent or transparent uniform shade.

Examples of pigments with special effects that may be mentioned, in a non-limiting manner, include white nacreous pigments such as mica coated with titanium or with bismuth oxychloride, colored nacreous pigments such as mica coated with titanium and with iron oxides, mica coated with titanium and for instance, with ferric blue or with chromium oxide, mica coated with titanium and with an organic pigment as defined above, and also nacreous pigments based on bismuth oxychloride.

Non-limiting mention may also be made of pigments with an interference effect not bound to a substrate, for instance liquid crystals (such as Helicones HC from Wacker), and holographic interference flakes (such as Geometric Pigments or Spectra f/x from Spectratek). The pigments with special effects can also be fluorescent pigments, whether they are substances that are fluorescent in daylight or that produce ultraviolet fluorescence, phosphorescent pigments, photochromic pigments, thermochromic pigments and quantum dots, for example sold by the company Quantum Dots Corporation.

Quantum dots are luminescent semiconductive nanoparticles capable of emitting, under light excitation, radiation with a wavelength ranging from 400 nm to 700 nm. These nanoparticles are known in the literature. For instance, they may be manufactured according to the processes described, for example, in U.S. Pat. Nos. 6,225,198 and 5,990,479, in the publications cited therein and also in the following publications: Dabboussi B. O. et al “(CdSe)ZnS core-shell quantum dots: synthesis and characterisation of a size series of highly luminescent nanocrystallites,” Journal of Physical Chemistry B, vol 101, 1997, pp 9463-9475 and Peng, Xiaogang et al, “Epitaxial Growth of highly Luminescent CdSe/CdS core/shell nanocrystals with photostability and electronic accessibility,” Journal of the American Chemical Society, vol 119, No. 30, pp 7019-7029.

The at least one pigment in accordance with the present disclosure can be, for example, chosen from colored pigments.

The variety of pigments that can be used makes it possible to obtain a rich palette of colors, and also, for example optical effects such as metallic or interference effects.

The size of the at least one pigment (other than the nacres) in solution can range from 10 nm to 10 μm, for instance from 50 nm to 5 μm, such as from 100 nm to 3 μm. The size of the at least one nacre in solution can range from 1 and 200 μm, for instance from 1 μm to 80 μm, such as from 1 μm to 50 μm.

According to one embodiment of the present disclosure, for example, the at least one pigment is in dispersion in the composition in accordance with the present disclosure.

The at least one polymer PA and at least one polymer PB can each be present in the composition in accordance with the present disclosure in an amount ranging from 0.05% to 50% by weight, for instance, from 0.1% to 20% by weight, such as from 0.25% to 10% by weight, relative to the total weight of the composition.

The at least one pigment can be present in the composition in accordance with the present disclosure, in an amount for each pigment, ranging from 0.05% to 80% by weight, for instance ranging from 0.1% to 60% by weight, such as from 0.25% to 40% by weight, relative to the total weight of the composition.

The composition according to the present disclosure may further comprise at least one activator. For example, the at least one activator can be chosen from pH modifiers, co-reagents and catalysts. Non-limiting examples include:
as pH modifier: acids or bases, of mineral or organic nature;
as co-reagent: carbodiimide, oxidizing agents or reducing agents;
as catalyst: enzymes such as transglutaminase.

According to another embodiment of the present disclosure, the at least one activator can be dispersed in the composition in accordance with the present disclosure.

The at least one activator, when present, can be present in an amount for each activator ranging from 0.05% to 30% by weight, for instance, from 0.1% to 20% by weight, such as from 0.25% to 10% by weight relative to the total weight of the composition.

The composition in accordance with the present disclosure may furthermore comprise at least one filler.

As used herein, the term “fillers” is understood to mean colorless or white, mineral or synthetic, lamellar or non-lamellar particles. The at least one filler may be present in an amount ranging from 0% to 80% by weight, for instance from 0.01% to 60% by weight, such as from 0.02% to 40% by weight, relative to the total weight of the composition. Non-limiting mention may be made of, for example, talc, zinc stearate, kaolin, polyamide (Nylon®) (Orgasol from Atochem) powders, polyethylene powders, powders of tetrafluoroethylene polymers (Teflon®), starch, boron nitride, polymer microspheres such as those of polyvinylidene chloride/acrylonitrile, for instance Expancel (Nobel Industrie), acrylic acid copolymer microspheres (Polytrap® from the company Dow Corning), silicone resin microbeads (for example Tospearls® from Toshiba) and elastomeric organopolysiloxanes.

The composition according to the present disclosure may also comprise at least one conventional cosmetic additive chosen from fixing polymers; thickeners; anionic, nonionic, cationic and amphoteric surfactants; fragrances; preserving agents; sunscreens; proteins; vitamins; provitamins; anionic, nonionic, cationic and amphoteric non-fixing polymers; mineral, plant and synthetic oils; ceramides; pseudoceramides; volatile and non-volatile, linear and cyclic, modified and unmodified silicones; pH regulators; oxidizing agents; reducing agents; inhibitors; catalysts; oxidation dyes such as oxidation bases and couplers; direct dyes other than pigments; and any other additive conventionally used in cosmetic compositions.

The process according to the present disclosure comprises applying to the keratin fibers:
at least one polymer PA and at least one polymer PB as defined above; and
at least one pigment as defined above.

The at least one polymer PA, the at least one polymer PB, and the at least one pigment can be applied to the keratin fibers using several compositions comprising the at least one polymer PA, the at least one polymer PB, and the at least one pigment, alone or as a mixture, or using only one composition comprising the at least one polymer PA, the at least one polymer PB, and the at least one pigment.

According to one embodiment of the present disclosure, a composition (A) comprising, in a cosmetically acceptable medium, at least one pigment, at least one polymer PA, and at least one polymer PB, is applied to the keratin fibers.

According to another embodiment of the present disclosure, a composition (B) comprising, in a cosmetically acceptable medium, at least one pigment, and a composition (C) comprising, in a cosmetically acceptable medium, at least one polymer PA and at least one polymer PB, are applied to the keratin fibers, the order of application of the compositions (B) and (C) being irrelevant.

According to still another embodiment of the present disclosure, a composition (B) comprising, in a cosmetically acceptable medium, at least one pigment, a composition (D) comprising, in a cosmetically acceptable medium, at least one polymer PA, and a composition (E) comprising, in a cosmetically acceptable medium, at least one polymer PB, are applied to the keratin fibers, the order of application of the compositions (B), (D) and (E) being irrelevant.

According to yet another embodiment of the present disclosure, a composition (F) comprising, in a cosmetically acceptable medium, at least one polymer PA, and at least one pigment, and a composition (E) comprising, in a cosmetically acceptable medium, at least one polymer PB, are applied to the keratin fibers, the order of application of the compositions (F) and (E) being irrelevant.

According to still further another embodiment of the present disclosure, a composition (D) comprising, in a cosmetically acceptable medium, at least one polymer PA, and a composition (G) comprising, in a cosmetically acceptable medium, at least one polymer PB and at least one pigment, are applied to the keratin fibers, the order of application of the compositions (D) and (G) being irrelevant.

According to one embodiment of the present disclosure, the composition comprising at least one pigment is applied before the at least one composition comprising the at least one polymer PA and/or the at least one polymer PB.

According to an additional embodiment of the present disclosure, the temperature is increased to activate the reaction between at least one polymer PA and at least one polymer PB. For example, the temperature may be increased to 180° C. or 200° C.

According to still yet another embodiment of the present disclosure, at least one activator as defined above is applied to the keratin fibers to activate the reaction between the at least one polymer PA and the at least one polymer PB.

For example, the at least one activator may be present in at least one of the compositions applied to the keratin fibers or in an additional composition, in which case the order of application of the various compositions to the keratin fibers is irrelevant.

The at least one activator can be chosen, for example, from those described above.

According to another embodiment of the present disclosure, the at least one pigment and the at least one activator are dispersed in the various compositions used in the process in accordance with the present disclosure comprising them.

The various compositions used in the process in accordance with the present disclosure may be applied to dry or wet hair.

These compositions may also comprise at least one conventional cosmetic additive as defined above.

Intermediate drying may be performed between each application.

When the reaction between the at least one polymer PA and at least one polymer PB takes place spontaneously at room temperature, but their mixture in dilute solution is stable, a solution comprising the polymers in at least one cosmetically acceptable volatile solvent may be applied directly to the keratin fibers, and the crosslinking reaction takes place in the course of evaporation of the at least one solvent. The polymer deposit becomes insoluble and remains on the keratin fibers.

If the at least one solvent used is not volatile, the keratin fibers may be dried under a hood, with a hairdryer or with smoothing tongs.

When the reaction between the at least one polymer PA and the at least one polymer PB requires activation, the polymer mixture may be applied to the keratin fibers and, by increasing the temperature, or by adding at least one pH modifier, or by adding at least one co-reagent or at least one catalyst, crosslinking of the deposit is brought about. This addition may take place immediately after the deposition of the polymers onto the keratin fibers, after predrying of the hair.

When the at least one polymer PA and at least one polymer PB are incompatible in solution, at least one of the two polymers is first deposited onto the keratin fibers via a cosmetically acceptable medium, and the at least one polymer that can react with the first is then deposited, via a cosmetically acceptable medium, with or without intermediate drying. The chemical reaction may then take place spontaneously during the drying or may be initiated by supplying heat, a change in pH or an addition of at least one co-reagent or of at least one catalyst. The order of application of the polymers may vary.

The keratin fibers may be dried under a hood, with a hairdryer or with smoothing tongs.

The crosslinked deposit thus formed can have the benefit of having an unexpected low solubility. In addition, it has good affinity for the surface of the keratin fibers, which ensures better remaining power of the deposit as a whole.

When the polymers are applied separately, the layered deposit obtained may also be beneficial for conserving the cosmetic or optical properties of the at least one polymer that comprises the upper part of the deposit.

According to the processes describe herein, it is possible to produce multiple superpositions of layers of polymers that crosslink together to achieve the desired type of deposit (in terms of chemical nature, mechanical strength, thickness, appearance, feel, etc.).

The cosmetically acceptable medium, conveying the at least one polymer PA and/or the at least one polymer PB, is chosen such that the at least one polymer PA and the at least one polymer PB are capable of reacting with each other to form covalent bonds after application of the cosmetic composition to the hair.

In the context of the present disclosure, a cosmetically acceptable medium can consist of water, or can comprise water and at least one cosmetically acceptable solvents such as alcohols, esters, ketones, and cyclic volatile silicones. In one embodiment, these solvents can be C₁-C₄ alcohols.

The present disclosure also relates to a multi-compartment device or “kit” comprising at least two compartments comprising at least two compositions such that the combination of the compositions comprises:
at least one polymer PA and at least one polymer PB as defined above; and
at least one pigment as defined above.

According to one embodiment of the present disclosure, at least one first compartment comprises at least one composition (B) as defined above and at least one second compartment comprises at least one composition (C) as defined above.

According to another embodiment, at least one first compartment comprises at least one composition (B) as defined above, at least one second compartment comprises at least one composition (D) as defined above and a third compartment comprises at least one composition (E) as defined above.

According to still another embodiment of the present disclosure, at least one first compartment comprises at least one composition (F) as defined above and at least one second compartment comprises at least one composition (E) as defined above.

According to yet another embodiment, at least one first compartment comprises at least one composition (D) as defined above and at least one second compartment comprises at least one composition (G) as defined above.

According to one embodiment of the present disclosure, the combination of the compositions also comprises at least one activator as defined above. For example, at least one of the compositions comprised in the multi-compartment kit may also comprise at least one activator.

The multi-compartment kit may also comprise at least one additional composition comprising, in a cosmetically acceptable medium, at least one activator.

The at least one activator can be chosen, for example, from those described above.

The present disclosure further relates to the use, for dyeing keratin fibers, of at least one pigment as defined above, of at least one polymer PA and of at least one polymer PB as defined above.

For instance, one aspect of the present disclosure is a method for making dyed keratin fibers color-fast, comprising applying to keratin fibers a composition comprising at least one pigment as defined above, at least one polymer PA, and at least one polymer PB as defined above, wherein the at least one pigment, at least one polymer PA, and at least one polymer PB are present in an amount effective to make the resulting coloration of the keratin fibers color-fast. For example, the color-fastness can be such that the percentage of degradation after six shampoo washes as defined below is less than or equal to 50%.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

The following examples were performed using the polymer couple PA, PB of Polylysine (polymer PA)-Gantrez (polymer PB). Accordingly, in this case it was a matter of crosslinking between an amine functional group and an aldehyde functional group.

The treated keratin fibers were locks of 1 g of natural hair.

The various reagents used were as follows:
Pigment 1: Composite pigment of 17 nm with a silica core and an organic part consisting of quinacridone (pigment Red 122) prepared via the method described in European Patent Application No. EP 1 184 426 A2;
Pigment 2: Organic quinacridone pigment, pigment Red 122 (sunfast magenta 122 sold by the group Sun);
Pigment 3: Calcium salt of lithol B red, D & C Red 7 sold by the company Wackherr;
Polylysine sold by Chisso;
Gantrez S95 sold by ISP;
EDC: N-(3-Dimethylaminopropyl)(N′-ethylcarbodiimide) hydrochloride sold by Fluka;
NHS: N-Hydroxysulfosuccinimide, sodium salt sold by Fluka.

The following solutions were prepared from these reagents:
Solution A₁: Aqueous 10% solution of pigment 1,
Solution A₂: Aqueous 10% solution of pigment 2,
Solution A₃: Aqueous 10% solution of pigment 3,
Solution B: Aqueous 10% solution of polylysine,
Solution C: Aqueous 5% solution of Gantrez S95,
Solution D₁: Aqueous solution containing 10% of pigment 1 and 10% of polylysine,
Solution D₂: Aqueous solution containing 10% of pigment 2 and 10% of polylysine,
Solution D₃: Aqueous solution containing 10% of pigment 3 and 10% of polylysine,
Solution E1: Aqueous solution containing 10% of pigment 1 and 5% of Gantrez,
Solution E₂: Aqueous solution containing 10% of pigment 2 and 5% of Gantrez, Solution E₃: Aqueous solution containing 10% of pigment 3 and 5% of Gantrez,
Solution F: Aqueous solution containing 6×10⁻⁵ mol of EDC and 6×10⁻⁵ mol of NHS.

### Example 1

1) 0.5 g of solution A₁ was applied to a lock of clean dry hair. The lock was then dried under a hood.
2) 0.5 g of solution B was then applied to the dry lock. Without intermediate drying, 0.5 g of solution C was applied.
3) The lock was then placed at 140° C. for 30 minutes.

The same procedure was performed for solutions A₂ and A₃.

With solutions A₁ and A₂, the locks obtained were strongly colored pink. With solution A₃, the lock obtained was strongly colored red. The locks were then subjected to a color-fastness test with respect to shampooing.

The color of the hair was measured using a Minolta CM3600d® spectrocolorimeter in the CIE Lab system. In this system, L* represents the luminance, a* represents the hue, and b* is the saturation.

The degradation of the color after six shampoo washes was estimated according to the following formula:

$$\% degradation = 100 * \frac{DE 6 Shamp}{DE Contr}$$

$$with$$

$$DE contr = \sqrt{{( {a}^{*} contr - {a}^{*} BN )}^{2} + {( {b}^{*} contr - {b}^{*} BN )}^{2} + {( {L}^{*} contr - {L}^{*} BN )}^{2}}$$

$$and$$

$$DE 6 Shamp = \sqrt{{( {a}^{*} contr - {a}^{*} 6 Sh )}^{2} + {( {b}^{*} contr - {b}^{*} 6 Sh )}^{2} + {( {L}^{*} contr - {L}^{*} 6 Sh )}^{2}}$$

in which a*BN, b*BN and L*BN are the values of a*, b* and L* for the uncolored lock, a*tem, b*tem and L*contr the values of a*, b* and L* for the colored lock before shampooing, while a*6Sh, b*6Sh and L*6Sh are the values of a*, b* and L* for the colored lock after 6 shampoo washes.

With solution A₁, the degradation of the color was 9.19% after 6 shampoo washes.

With solution A₂, the degradation of the color was 13.69% after 6 shampoo washes.

With solution A₃, the degradation of the color was 19.69% after 6 shampoo washes.

These results show that the coloration obtained with the process in accordance with the present disclosure shows good shampoo-fastness.

### Example 2

1) 0.5 g of solution A₁ was applied to a lock of clean dry hair. The lock was then dried under a hood.
2) 0.5 g of solution C was then applied to the dry lock. Without intermediate drying, 0.5 g of solution B was applied.
3) The lock was then placed at 140° C. for 30 minutes.

The same procedure was performed for solutions A₂ and A₃.

With solutions A₁ and A₂, the locks obtained were strongly colored pink. With solution A₃, the lock obtained was strongly colored red.

### Example 3

1) 0.5 g of solution A₁ was applied to a lock of clean dry hair. The lock was then dried under a hood.
2) 0.5 g of solution B was then applied to the dry lock. Without intermediate drying, 0.5 g of solution C was applied.
3) The lock was then dried with smoothing tongs at 180° C. by pressing on each part of the lock for 30 seconds.

The same procedure was performed for solutions A₂ and A₃.

With solutions A₁ and A₂, the locks obtained were strongly colored pink. With solution A₃, the lock obtained was strongly colored red.

### Example 4

1) 0.5 g of solution D₁ was applied to a lock of clean dry hair. Without intermediate drying, 0.5 g of solution C was applied.
2) The lock was then placed at 140° C. for 30 minutes.

The same procedure was performed for solutions D₂ and D₃.

With solutions D₁ and D₂, the locks obtained were strongly colored pink. With solution D₃, the lock obtained was strongly colored red.

### Example 5

1) 0.5 g of solution E₁ was applied to a lock of clean dry hair. Without intermediate drying, 0.5 g of solution B was applied.
2) The lock was then placed at 140° C. for 30 minutes.

The same procedure was performed for solutions E₂ and E₃.

With solutions E₁ and E₂, the locks obtained were strongly colored pink. With solution E₃, the lock obtained was strongly colored red.

### Example 6

1) 0.5 g of solution A₁ was applied to a lock of clean dry hair. The lock was then dried under a hood.
2) 0.5 g of solution B was then applied to the dry lock. Without intermediate drying, 0.5 g of solution C was applied.
3) The lock was then placed under a hood for 2 minutes.
4) 0.3 g of a freshly prepared (less than 5 minutes) solution F was applied to the lock. This lock was then dried with a hairdryer or smoothing tongs.

The same procedure was performed for solutions A₂ and A₃.

With solutions A₁ and A₂, the locks obtained were strongly colored pink. With solution A₃, the lock obtained was strongly colored red.

1. A composition for dyeing keratin fibers, comprising, in a cosmetically acceptable medium, at least one pigment, at least one polymer PA bearing at least one chemical functional group A, and at least one polymer PB bearing at least one chemical functional group B, wherein the chemical functional groups A and B can form covalent bonds together.

2. The composition according to claim 1, wherein the at least one chemical functional group A is chosen from:
epoxide,
aziridine,
vinyl and activated vinyl,
carboxylic acid anhydride, acid chloride and esters,
aldehydes,
acetals, hemiacetals,
aminals, hemiaminals,
ketones, α-hydroxy ketones, α-halo ketones,
lactones, thiolactones,
isocyanate,
thiocyanate,
imines,
imides,
N-hydroxysuccinimide esters,
imidates,
thiosulfate,
oxazine and oxazoline,
oxazinium and oxazolinium,
C₁ to C₃₀ alkyl halides or C₆ to C₃₀ aryl or aralkyl halides of formula RX wherein X is chosen from I, Br and Cl,
halides of a carbon-based unsaturated ring or an unsaturated heterocycle,
sulfonyl halides of formula RSO₂Cl or RSO₂F, wherein R is chosen from C₁ to C₃₀ alkyl groups.

3. The composition according to claim 2, wherein the at least one chemical functional group A is chosen from anhydride, epoxide, chlorotriazine, aldehyde and thiosulfate functional groups.

4. The composition according to claim 1, wherein the at least one chemical functional group B is chosen from hydroxyl, primary and secondary amine, thiol and carboxylic acid functional groups.

5. The composition according to claim 1, wherein the at least one polymer PA bears at least two identical chemical functional groups A, and the at least one polymer PB bears at least two identical chemical functional groups B.

6. The composition according to claim 1, wherein the at least one polymer PA is chosen from:
polymers comprising an anhydride unit,
polymers comprising an epoxide and/or aldehyde group,
natural and modified polysaccharides comprising aldehyde functional groups,
natural and modified polysaccharides comprising epoxy functional groups, and
natural and synthetic polymers comprising carboxylic acid functional groups.

7. The composition according to claim 1, wherein the at least one polymer PB is chosen from natural and synthetic polymers comprising OH, NH₂, SH or COOH functional groups.

8. The composition according to claim 7, wherein the natural and synthetic polymers comprising OH, NH₂, SH or COOH functional groups are chosen from:
dendrimers comprising OH, NH₂, SH or COOH end groups of at least one generation,
synthetic polymers comprising a hydroxyl functional group,
polyethyleneimines,
polyethyleneimine thiols obtained by reacting polyethyleneimines with γ-butyrolactone,
polyamino acids comprising free OH, NH₂, SH or COOH groups, and
natural and modified polysaccharides comprising OH, NH₂, SH or COOH functional groups.

9. The composition according to claim 1, wherein the at least one polymer PA and the at least one polymer PB are chosen from the following combinations:
at least one polymer PB chosen from dendrimers comprising OH, NH₂, SH or COOH end groups of at least one generation in combination with at least one polymer PA chosen from polymers comprising an anhydride unit or polymers comprising an epoxide and/or aldehyde group,
at least one polymer PB chosen from synthetic polymers comprising a hydroxyl functional group in combination with at least one polymer PA chosen from polymers comprising an anhydride unit or polymers comprising an epoxide group,
at least one polymer PB chosen from polyethyleneimines in combination with at least one polymer PA chosen from polymers comprising an anhydride unit or polymers comprising an epoxide and/or aldehyde group,
at least one polymer PB chosen from polyethyleneimine thiols, obtained by reacting polyethyleneimines with γ-butyrolactone, in combination with at least one polymer PA chosen from polymers comprising an anhydride unit or polymers comprising an epoxide and/or aldehyde group,
at least one polymer PB chosen from polyamino acids comprising free OH, NH₂, SH or COOH groups in combination with at least one polymer PA chosen from polymers comprising an anhydride unit or polymers comprising an epoxide and/or aldehyde group,
at least one polymer PB chosen from natural or modified polysaccharides comprising OH, NH₂, SH or COOH functional groups in combination with at least one polymer PA chosen from polymers comprising an anhydride unit or polymers comprising an epoxide and/or aldehyde group,
at least one polymer PA chosen from natural or modified polysaccharides comprising aldehyde functional groups in combination with at least one polymer PB chosen from synthetic or natural polymers comprising OH, NH₂, SH or COOH functional groups,
at least one polymer PA chosen from natural or modified polysaccharides comprising epoxy functional groups in combination with at least one polymer PB chosen from synthetic or natural polymers comprising OH, NH₂, SH or COOH functional groups, and
at least one polymer PA chosen from natural or synthetic polymers comprising carboxylic acid functional groups in combination with at least one polymer PB chosen from synthetic or natural polymers comprising OH, NH₂ or SH functional groups, in the presence of a carbodiimide, or of an acid, a base or an enzyme.

10. The composition according to claim 1, wherein the at least one polymer PA bearing the at least one chemical functional group A is chosen from:
methyl vinyl ether/maleic anhydride copolymer,
polyglycidyl methacrylate,
glycidyl polydimethylsiloxane,
epoxy polyamidoamine,
epoxydextran, and
polyaldehyde polysaccharides obtained by oxidation of polysaccharides with NaIO₄.

11. The composition according to claim 1, wherein the at least one polymer PB bearing the at least one chemical functional group B is chosen from:
PAMAM dendrimer,
dendrimer comprising hydroxyl functional groups,
PEI (polyethyleneimine),
PEI-thiol,
polylysine,
HP cellulose,
aminodextran,
aminocellulose,
PVA (polyvinyl acetal),
Amino PVA,
chitosan,
CM or HP dextran, and
CM or HP chitosan.

12. The composition according to claim 1, wherein the at least one pigment is in a form chosen from powder and pigmentary paste.

13. The composition according to claim 12, wherein the at least one pigment is a mineral pigment chosen from titanium dioxide, which may or may not be surface-treated, zirconium oxide, cerium oxide, iron oxide, chromium oxide, manganese violet, ultramarine blue, chromium hydrate and ferric blue.

14. The composition according to claim 12, wherein the at least one pigment is an organic pigment chosen from nitroso, nitro, azo, xanthene, quinoline, anthraquinone and phthalocyanin compounds; compounds of metallic complex type; and isoindolinone, isoindoline, quinacridone, perinone, perylene, diketopyrrolopyrrole, thioindigo, dioxazine, triphenylmethane and quinophthalone compounds.

15. The composition according to claim 12, wherein the at least one pigment is a composite pigment composed of particles comprising:
a mineral core,
at least one binder for fixing the organic pigments to the core, and
at least one organic pigment at least partially covering the core.

16. The composition according to claim 12, wherein the at least one pigment is a lake comprising a mineral substrate chosen from alumina, silica, calcium sodium borosilicate, calcium aluminium borosilicate, and aluminium on which a dye is adsorbed.

17. The composition according to claim 12, wherein the at least one pigment is chosen from pigments with special effects chosen from nacreous pigments, pigments with interference effects not bound to a substrate, fluorescent pigments, phosphorescent pigments, photochromic pigments, thermochromic pigments, and quantum dots.

18. The composition according to claim 17, wherein the at least one nacreous pigment is chosen from mica coated with titanium, mica coated with bismuth oxychloride, mica coated with titanium and iron oxides, mica coated with titanium and with ferric blue or chromium oxide, mica coated with titanium and with at least one organic pigment chosen from nitroso, nitro, azo, xanthene, quinoline, anthraquinone and phthalocyanin compounds; compounds of metallic complex type; and isoindolinone, isoindoline, quinacridone, perinone, perylene, diketopyrrolopyrrole, thioindigo, dioxazine, triphenylmethane and quinophthalone compounds, and nacreous pigments based on bismuth oxychloride.

19. The composition according to claim 17, wherein the at least one pigment with an interference effect not bound to a substrate is chosen from liquid crystals and holographic interference flakes.

20. The composition according to claim 1, wherein the at least one pigment is chosen from colored pigments.

21. The composition according to claim 1, wherein the at least one pigment is in dispersion.

22. The composition according to claim 1, wherein the at least one polymer PA and the at least one polymer PB are each present in an amount ranging from 0.05% to 50% by weight, relative to the total weight of the composition.

23. The composition according to claim 1, wherein the at least one pigment is present in an amount, for each pigment present, ranging from 0.05% to 80% by weight, relative to the total weight of the composition.

24. The composition according to claim 1, further comprising at least one activator.

25. The composition according to claim 24, wherein the at least one activator is chosen from pH modifiers, co-reagents and catalysts.

26. The composition according to claim 24, wherein the at least one activator is in dispersion.

27. The composition according to claim 24, wherein the at least one activator is present in an amount, for each activator present, ranging from 0.05% to 30% by weight, relative to the total weight of the composition.

28. A process for dyeing keratin fibers, comprising applying to the keratin fibers:
at least one pigment
at least one polymer PA bearing at least one chemical functional group A, and
at least one polymer PB bearing at least one chemical functional group B, wherein the chemical functional groups A and B can form covalent bonds together.

29. The process according to claim 28, wherein the at least one pigment, the at least one polymer PA, and the at least on polymer PB are applied to the keratin fibers using several compositions comprising the at least one pigment, the at least one polymer PA, and the at least one polymer PB, alone or as a mixture, or using only one composition comprising the at least one pigment, the at least one polymer PA, and the at least one polymer PB.

30. The process according to claim 29, wherein the composition comprising the at least one pigment is applied before the at least one composition comprising the at least one polymer PA and/or the at least one polymer PB.

31. The process according to claim 28, wherein the keratin fibers are exposed to an increase in temperature to activate the reaction between the at least one polymer PA and the at least one polymer PB.

32. The process according to claim 28, wherein at least one activator is also applied to the keratin fibers to activate the reaction between the at least one polymer PA and the at least one polymer PB.

33. A multi-compartment kit, comprising at least two compartments comprising at least two compositions such that the combination of the at least two compositions comprises:
at least one pigment,
at least one polymer PA bearing at least one chemical functional group A, and
at least one polymer PB bearing at least one chemical functional group B, wherein the chemical functional groups A and B can form covalent bonds together.

34. The kit according to claim 33, wherein the combination of the at least two compositions further comprises at least one activator.

35. A method for making dyed keratin fibers color-fast, comprising applying to the keratin fibers at least one composition comprising at least one pigment, at least one polymer PA bearing at least one chemical functional group A, and at least one polymer PB bearing at least one chemical functional group B, wherein the chemical functional groups A and B can form covalent bonds together, and
wherein the at least one pigment, at least one polymer PA, and at least one polymer PB are present in an amount effective to make the resulting coloration of the keratin fibers color-fast.

36. The method according to claim 35, wherein the coloration of the keratin fibers is color-fast with respect to shampoo.
